(19) United States
(12) Patent Application Publication
(10) Pub. No.: US 2008/0131918 A1
(43) Pub. Date: Jun. 5, 2008

(54) Cholesterol Assay

(75) Inventors: Gareth Royston Jones, Altrincham (GB); David Thomas Clarke, Warrington (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Science And Technology Facilities Council, Warrington (GB)

(21) Appl. No.: 11/792,875

(22) PCT Filed: Dec. 12, 2005
(86) PCT No.: PCT/GB05/04759

§ 371 Date: Jun. 7, 2007

(30) Foreign Application Priority Data
Dec. 11, 2004 (GB) 0427189.6

(51) Int. Cl. G01N 33/92; C12Q 1/60; G01N 21/76

(52) U.S. Cl. 435/11; 436/71; 422/52

(57) ABSTRACT

The present invention relates to a method of determining the concentration of cholesterol in a sample (e.g. a blood sample). The method comprising the steps of adding to the sample reagents which cause the Liebermann-Burchard (L-B) reaction; and then determining the total cholesterol concentration in the sample using fluorescence analysis. The invention also relates to apparatus, and particularly apparatus in the form of a reader and cartridge, for conducting the method of the invention.

The present invention relates to an assay system for discriminating between different classes of lipid molecules in a sample mixture. In particular, the invention relates to a method of determining the concentration of cholesterol in blood plasma or serum. The invention further relates to an apparatus for carrying out the method.

Lipids are a diverse group of organic compounds occurring in living organisms. They are insoluble in water, but soluble in organic solvents. Lipids are broadly classified into two categories: (i) complex lipids; and (ii) simple lipids. Complex lipids are esters of long-chain fatty acids and include glycerides, glycolipids, phospholipids, cholesterol esters and waxes. Simple lipids, which do not contain fatty acids, include steroids (for example, cholesterol) and terpenes.

Lipids can combine with proteins to form lipoproteins, which is the form in which lipids, such as cholesterol and triglycerides, are transported in blood and lymph. For brevity, the term “serum” is used herein, but references to “serum” should be interpreted as references to serum or plasma. The lipoproteins found in blood plasma fall into three main classifications: (i) high density lipoproteins (HDL), (ii) low density lipoproteins (LDL), and (iii) very low density lipoproteins (VLDL) together with intermediate density lipoproteins (IDL).

Cholesterol is mainly transported in the bloodstream in the form of LDLs, and is removed from the blood by means of LDL receptors in the liver. The LDLs containing the cholesterol are bound to the LDL receptors, which are then taken into cells. Lack of LDL receptors, occurring as a genetic defect in some individuals, is believed to be a cause of high levels of cholesterol in the blood, predisposing them to atherosclerosis, i.e. the development of harmful plaques on blood vessel walls, which can lead to a heart attack and stroke. It is well documented that there is a strong relationship between the concentration of the various lipoproteins in blood plasma and the risk of atherosclerosis. It is also known that the different classes of lipoproteins (HDL, LDL and VLDL) each play a different role in atherosclerosis. For instance, HDL is regarded as being anti-atherogenic whereas LDL is known to be highly atherogenic (the cholesterol it carries correlating closely with atheroscleroses development). VLDL is considered to be slightly atherogenic, and of more significance in females.

Therefore, knowledge of the relative concentrations of each of the various lipid components in the blood, in particular, cholesterol, would be advantageous, as this would assist a clinician in treating patients having blood concentrations of these lipids, which are inappropriate.

Assays have been developed for determining the concentrations of some of the lipid components in blood. Such assays normally involve initially taking a blood sample from a patient, which is then sent to a clinical lab for analysis. Such assays have to be carried out using expensive equipment and reagents, and take a considerable length of time to generate results. This delays treatment. Furthermore, the tests are involved and are therefore expensive. In addition, the equipment used in the lab is not readily portable and so cannot be used by GPs, or nurses, carrying out house calls, or even as test kits for home use. Accordingly, there is a requirement to provide improved methods for analysing the lipid concentration in blood sera, and particularly of cholesterol.

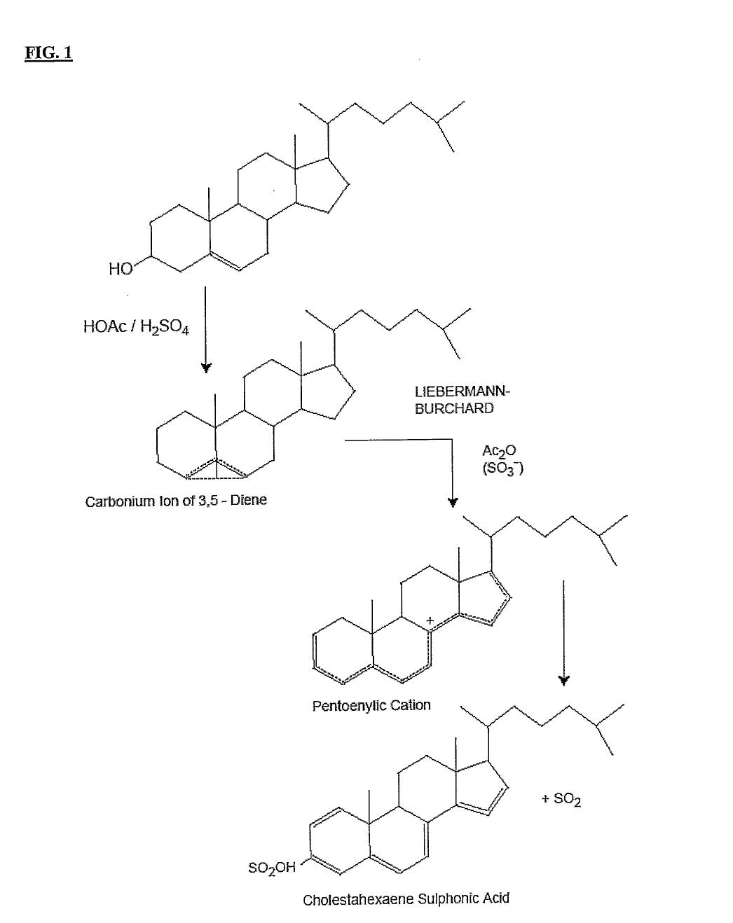

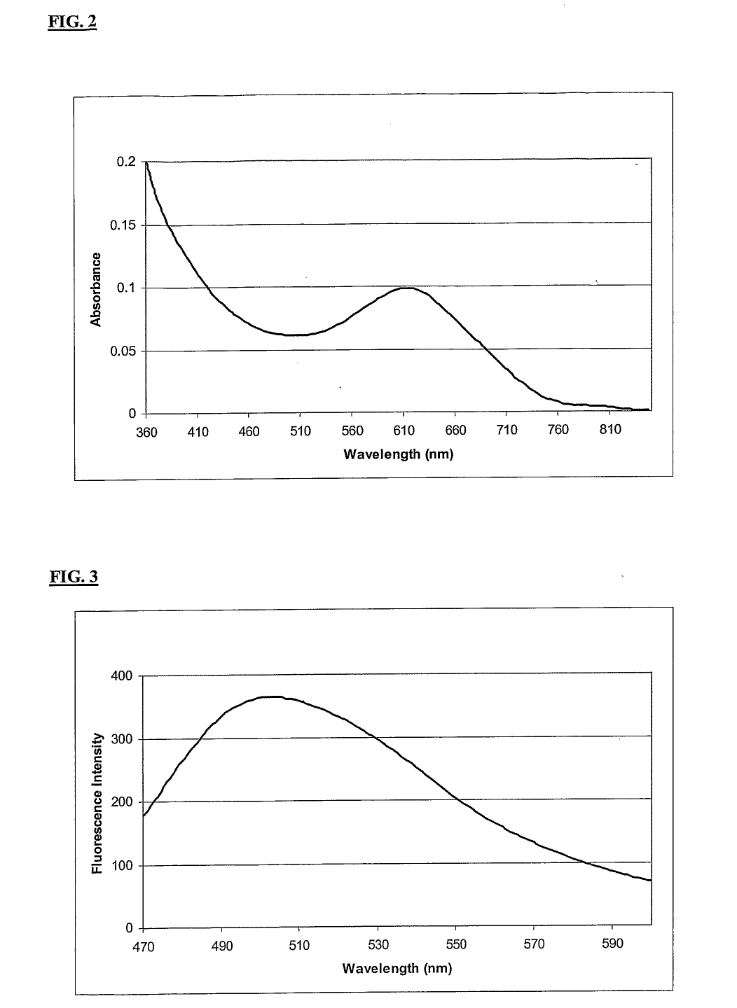

The Liebermann-Burchard (L-B) reaction assay is a well-known method for the measurement of total cholesterol in blood, and is considered to be the ‘gold standard’. This is an absorbance-based assay, and FIG. 1 shows a schematic for this Liebermann-Burchard reaction. First, the L-B reaction reagent is prepared, which consists of solution of 30% glacial acetic acid, 60% acetic anhydride, and 10% sulphuric acid. Secondly, 5 ml of this L-B reagent is then added to 0.2 ml of a sample derived from blood plasma, which are mixed together and then allowed to stand for 20 minutes. The L-B reaction is usually carried out on a sample comprising cholesterol that has been extracted from plasma into an organic solvent. The products of the L-B reaction are two coloured products. A typical absorbance spectrum for the product of the Liebermann-Burchard reaction is shown in FIG. 2. The absorbance of the products, the concentration of which are related to the concentration of cholesterol, is then measured using a spectrophotometer. The total concentration of cholesterol may be determined from a calibration curve of absorbance against cholesterol concentration, using cholesterol standards (Burke et al., Clin. Chem. 20 (7), 794-801 (1974)).

However, problems with the L-B reaction assay are that it requires relatively large quantities of reagents, which is a distinct disadvantage, as the reagents are very corrosive and require special care. It is also normally required that cholesterol is extracted from plasma and this extraction step constitutes a cumbersome extra step in the assay. Accordingly, the L-B reaction assay has been superseded in many laboratories by enzyme-linked assays, because of the requirement for fairly large sample quantities and the use of the corrosive reagents in the L-B reaction assay. However, use of such enzyme-linked assays for determining the concentration of total cholesterol, tend to be easier and safer to carry out, but are less accurate than the L-B assay. Because the results generated are less accurate, clinicians would prefer L-B reaction assay accuracy especially when determining a course of treatment for individuals with higher coronary heart disease risk factors.

Therefore, even though there are methods available for determining the concentration of cholesterol in blood samples, it will be appreciated that these methods have a number of limitations.

It is therefore an aim of embodiments of the present invention to obviate or mitigate the problems with the prior art, and to provide improved methods for determining the concentration of cholesterol in a sample taken from a subject. It is a further aim to provide an apparatus for carrying out said method.

As mentioned previously, the conventional L-B reaction assay measures absorbance, and therefore requires relatively large volumes of the L-B reaction reagents. For example, cuvettes used for measuring absorbance tend to hold about 1 ml of a sample to function. It will be appreciated that the components of the L-B reagents are very corrosive, which therefore require special care when used. The L-B assay is therefore quite onerous to use by lab technicians for measuring cholesterol concentrations in a blood sample.

Therefore, the inventors carried out some investigations with the reagents of the L-B assay to see whether or not it was possible to provide an improved assay for determining the concentration of cholesterol in a blood sample so that they were easier to use, less dangerous, and also more accurate. From their investigations, they were very surprised to find that the product of the L-B reaction actually fluoresced, and FIG. 3 shows the fluorescence emission spectrum of the L-B product. It was surprisingly observed that the fluorescence extends over the range of 470-600 nm.

Therefore, according to a first aspect of the present invention, there is provided a method of determining the concentration of total cholesterol in a sample, the method comprising the steps of:—
(i) adding to the sample reagents which cause the Liebermann-Burchard (L-B) reaction; and
(ii) determining the total cholesterol concentration in the sample using fluorescence analysis.

By the term “total cholesterol”, we mean the total concentration of cholesterol in the sample, which includes all cholesterol bound to carrier molecules such as lipoproteins, for example, LDL, and also any free cholesterol that may be present in the sample.

The sample may be a foodstuff, which requires analysis of the total cholesterol concentration therein. Preferably, the sample is a biological sample, which may be obtained from a subject to be tested. The sample may comprise any biological fluid, for example, blood serum or plasma, or lymph. It is especially preferred that the sample comprises blood serum. Another benefit of the method of the invention is that liquid samples (e.g. serum or plasma) may be used directly in the method. This is in contrast to a conventional L-B reaction assay that may require extraction of cholesterol from the primary sample before it is combined with the L-B reagents.

The Liebermann-Burchard (L-B) reaction for cholesterol is well-known, and a schematic showing the L-B reaction is illustrated in FIG. 1. Preferably, the reagents added to the sample result in the reaction of all of the total cholesterol present in the sample, i.e. the cholesterol and esters thereof, which may be associated with lipoproteins (e.g. LDL or HDL), which may be present in the sample. The L-B reagents preferably add double bonds to cholesterol in the sample, as is illustrated in FIG. 1. Accordingly, by the term “reagents which cause the L-B reaction”, we mean reagents which, when added to a sample containing cholesterol, cause or induce the cholesterol in the sample to be increasingly unsaturated.

The method according to the first aspect of the invention uses the same reagents as the generic Liebermann-Burchard reaction assay (L-B), or other assays for determining the concentration of cholesterol, which may be based on the L-B reaction, for example, the Abell-Kendal assay, which will be known to the skilled technician (Abell et al., J. Biol. Chem. 195 (1) p 357-366). However, instead of measuring the absorbance of the coloured products at 550 nm as in the conventional L-B reaction assay, the method according to the invention comprises measuring fluorescence to determine the cholesterol concentration in the sample. Hence, by the term “fluorescence analysis”, we mean the measurement of fluorescence of the products of the L-B reaction.

The L-B reaction reagents may comprise three different reagents,

The first reagent comprises a cholesterol solvent. Examples of suitable cholesterol solvents include acetic acid, dioxane, and/or chloroform. Preferably, the cholesterol solvent comprises glacial acetic acid.

The second L-B reaction reagent is a strong acid. The inventors do not wish to be bound by any hypothesis but believe the acid performs an elimination reaction that extracts water from cholesterol leaving a higher degree of conjugation. The inventors believe it is this conjugation (i.e. an increased number of double bonds) that fluoresces when excited according to the invention. The strong acid is preferably a oxo acids (X—OH) such as phosphoric acid (H₃PO₄) and more preferably H₂SO₄. The acid may also be HNO₃, H₂SeO₄, HClO₄, and HMnO₄. Alternatively the acid may be a lewis acid such as Al₂Cl₆, SnCl₄ and FeCl₃ or titanium dioxide.

It is most preferred that the strong acid comprises sulphuric acid, preferably, at a concentration of about 3-20% (v/v), or Al₂Cl₆ of about 0.5-2.5 Molar.

The third L-B reaction reagent comprises acetic anhydride. It is preferred that the acetic anhydride to solvent ratio is between 0.25:1 to 10:1, more preferably, between 0.5:1 to 5:1, and even more preferably, between 1:1 to 3:1. In a preferred embodiment, the acetic anhydride to solvent ratio is about 2:1.

It is preferred that the L-B reagents comprise about 30% (v/v) glacial acetic acid, about 60% (v/v) acetic anhydride, and about 10% (v/v) sulphuric acid.

In addition, the L-B reagents may also comprise additives such as, anhydrous sodium sulphate, or sodium salicylate etc., which are used to stabilise impurities in the sample. The additives may be added in the range of about 0.5-3% (v/v).

Preferably, the method comprises mixing the reagents with the sample in the assay. The method preferably comprises the step of exciting the sample (i.e. the product of the L-B reaction) at an excitation wavelength below about 500 nm, and more preferably, below about 470 nm. An especially preferred excitation wavelength of 450 nm or shorter wavelengths may be used in order to cause the product of the L-B reaction to fluoresce.

The method preferably further comprises the step of observing the emitted fluorescence at an emission wavelength of between 500-650 nm, and more preferably, between 520-600 nm. An especially preferred emission wavelength of 540 nm may be used, at which the most accurate readings for determining the cholesterol concentration may be observed.

It will be appreciated that measuring fluorescence is a considerable improvement over using the conventional L-B absorbance-based assay. Advantages of measuring the fluorescence of the product of the L-B reaction, instead of measuring its absorbance as in conventional methods, include the fact that much smaller volumes of the reagents are required. This is particularly advantageous as the reagents of the L-B reaction are very corrosive and therefore dangerous to use. Hence, reducing the amount of reagents required in the fluorescence assay of the method according to the first aspect is much safer for a laboratory technician, or other user, than is the case for the conventional L-B absorbance assay. Furthermore, using smaller volumes of reagents also means that a smaller device can be used for carrying out the assay.

In addition, measuring fluorescence is much more sensitive than measuring absorbance, and also much quicker to carry out. Therefore, it is possible to make a more accurate determination of the cholesterol concentration using fluorescence than measuring absorbance. Therefore, it will be appreciated that the method according to the invention may be used to quickly and accurately determine the concentration of total cholesterol in the sample, and is therefore a considerable improvement over the prior art.

Hence, a preferred method consists of an improved L-B reaction assay, which can be carried out to determine the concentration of cholesterol very quickly. A clinician may then use this information to decide upon a certain course of treatment. Therefore, a preferred embodiment of the invention may comprise taking a blood sample from a patient, and then separating the blood serum from the red blood cells. This may be achieved by known techniques, such as centrifugation or filtration. The serum may then be separated into an aliquot of about 1 ml in volume or less, which is then subjected to fluorescence analysis to determine the concentration of cholesterol therein.

The L-B reaction reagents may be added to the aliquot. It may then be excited at 450 nm in order to cause the product of the L-B reaction to fluoresce. This fluorescence may then be measured at an emission wavelength of 540 nm. From this value, it is then possible to determine the concentration of cholesterol in the sample.

In addition, to developing the method according to the first aspect, the inventors have also developed an apparatus for carrying out the method.

Hence, according to a second aspect of the present invention, there is provided apparatus for determining the concentration of total cholesterol in a sample, the apparatus comprising a reaction reservoir for conducting an L-B assay; containment means adapted to contain reagents required for the method according to the first aspect; means for mixing the sample and reagents in the reservoir; excitation means operable to excite the sample so that it fluoresces, and detection means operable to detect the fluorescence emitted by the sample.

Preferably, the apparatus comprises a number of reservoirs. A first reservoir may be for containing the sample and is referred to herein as a sample reservoir. The containment means may comprise a second reservoir, a reagent reservoir, for containing the L-B reagents. The reaction reservoir may be a third reservoir in which the assay to determine the concentration of total cholesterol in the sample may be conducted (following introduction of the sample and reagents from the respective first and second reservoirs). It is preferred that the reaction reservoir is arranged so that it may be excited by the excitation means. It is preferred that the reaction reservoir is arranged so that fluorescence produced from the assay (the L-B reaction) may be detected by the detection means.

It will be appreciated that, in some embodiments, the device may be designed such that the sample may be directly introduced into the reaction reservoir. This would obviate the need for a first or sample reservoir.

The reaction reservoir may comprise, or be connected to, containment means in which reagents of the L-B reaction may be contained. The reagents may comprise a cholesterol solvent system, for example, glacial acetic acid, and in addition, acetic anhydride, and preferably, sulphuric acid. It will be appreciated that these L-B reaction reagents are corrosive.

The apparatus may comprise a reader and preferably, a cartridge adapted to be placed in functional communication therewith. The reader may comprise the excitation and detection means whereas the cartridge may comprise the reaction reservoir and the containment means for the L-B reagents as-well-as any other reservoir referred to above. Preferably, the cartridge may be inserted into, or attached to, the reader. The reader may comprise docking means in which the cartridge may be inserted. The docking means may be a slot. Hence, preferably, the cartridge is removable from the reader. A user of the apparatus may insert the sample into a sample reservoir in the cartridge. The cartridge may comprise a containment means in the form of a reagent reservoir that is pre-loaded with the reagents. When the cartridge is inserted into the reader the sample and reagents may be urged into a reaction reservoir in the cartridge that is aligned with the excitation means and detection means in the reader. The fluorescence may then be read from the sample. Accordingly, advantageously, use of the cartridge means that the user does not need to handle or come in to contact with the corrosive L-B reaction reagents.

The cartridge may comprise the reservoir, and preferably, the containment means. Hence, the cartridge carrying the L-B reaction reagents may be removed from the reader once the reagents have been exhausted, and replaced with a new cartridge containing new (unused) L-B reaction reagents.

Preferably, the apparatus comprises processing means adapted to determine the concentration of total cholesterol in the sample based on the fluorescence detected. The apparatus may comprise display means for displaying the concentration of total cholesterol in the sample, preferably as a read-out. For example, the display means may comprise an LCD screen or may rely on a computer for powering, and/or computing, and/or display.

Preferably, the apparatus is portable, and may be used to calculate the concentration of cholesterol in a patient by a taking a sample therefrom. The sample may be any biological fluid, for example, blood, plasma, lymph etc. or a foodstuff.

Preferably, the excitation means comprises an illumination source operable to illuminate the sample at about 400 nm-500 nm. The illumination source may comprise a bulb or an LED. The excitation means may comprise a 450 nm interference filter. The excitation means may comprise polarising means operable to polarise light produced by the illumination source. The excitation means may comprise focussing means adapted to focus the light on to the sample. The focussing means may comprise a lens.

Preferably, the detection means comprises a photodiode or photomultiplier etc, which is preferably red sensitive. Fluorescence emitted by the sample is preferably detected at about 500 nm-650 nm, and more preferably, 540 nm. The fluorescence may be collected by a second lens, and may pass through a polariser. Scattered excitation light may be removed by a cut-off filter. For measurement of the fluorescence intensity, the current from the photodiode or the count rate from the photomultiplier may be read from an ammeter, voltmeter, or rate-meter module.

The apparatus may comprise an excitation correction system so that fluctuations of the light source may be accounted for. The apparatus may comprise at least one fluorescence standard for use in calibrating prior to calculating the concentration of cholesterol.

The apparatus may be designed to draw power (e.g. to power the LCD and any processors contained therein) from a battery incorporated therein. Alternatively power may be drawn from a computer (e.g. via a USB connection to a laptop computer or PDA) or even from a mobile phone.

Accordingly, the apparatus is configured to detect and measure the fluorescence intensity of the assay as the cartridge enters the reader or at some time thereafter, to thereby determine the concentration of cholesterol in the sample.

Advantageously, the apparatus according to the second aspect may be used to carry out quick and easy assays, which can be conducted to determine the concentration of cholesterol in the biological fluid. The clinician can then decide on an effective course of treatment. In addition, the apparatus is portable and may be used by GPs, or nurses who carry out homes visits, or even as test kits for home use.

It will be appreciated that knowledge of the method according to the first aspect of the invention enabled the inventors to design small, preferably portable, apparatus utilising small volumes of sample and reagents. It is preferred that the apparatus comprises a reader and cartridge. Such cartridges represent an important feature of the invention. Therefore according to a third aspect of the invention there is provided a cartridge comprising a reaction reservoir for conducting an L-B assay; containment means adapted to contain reagents required for the method according to the first aspect of the invention.

The cartridge should be adapted such that the reaction reservoir may be aligned with a reader to allow fluorescence measurements to be made from any sample contained within the reaction reservoir.

It is preferred that the cartridge further comprises a sample reservoir; that the containment means is in the from of a reagent reservoir; and also has channels connecting the sample reservoir and reagent reservoir to the reaction reservoir.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 shows a schematic for the Liebermann-Burchard reaction;

FIG. 2 shows the absorbance spectrum for the product of the Liebermann-Burchard reaction;

FIG. 3 shows the fluorescence emission spectrum for the product of the Liebermann-Burchard reaction;

FIG. 4 is a graph showing fluorescence intensity against the concentration of cholesterol as referred to in Example 1;

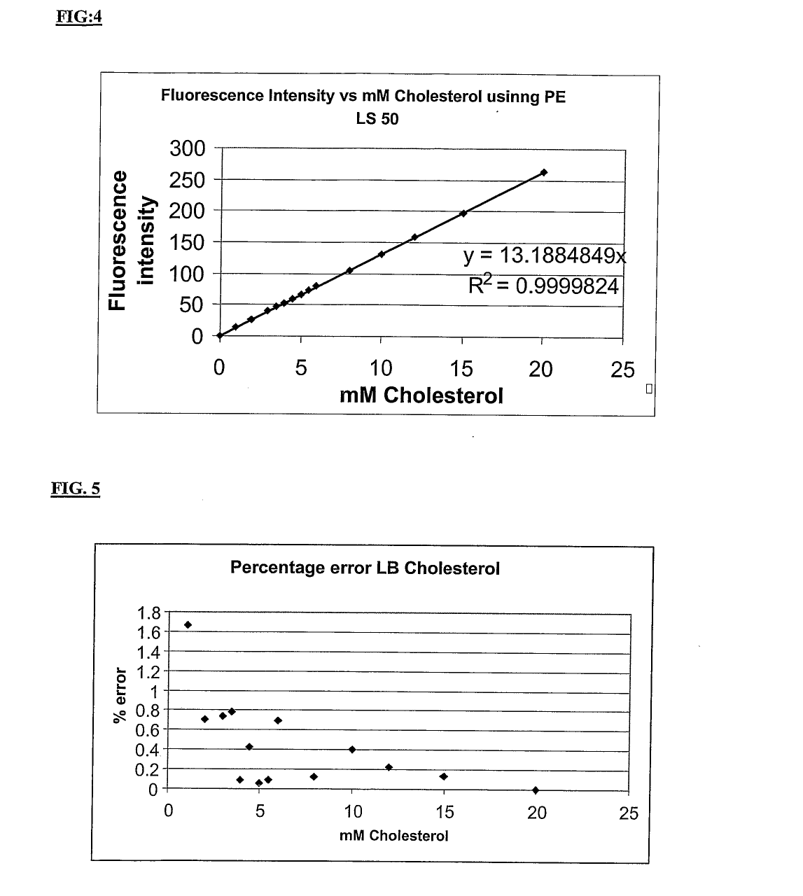

FIG. 5 is a graph showing percentage error for determining the concentration of cholesterol as referred to in Example 1;

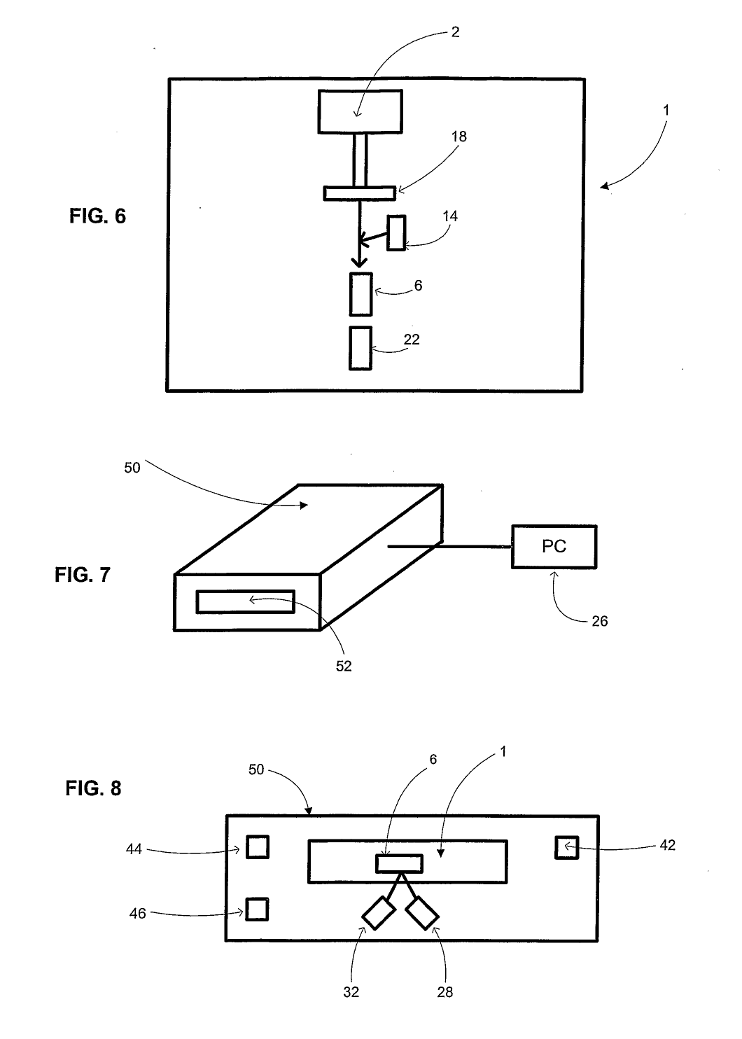

FIG. 6 shows a schematic view of an embodiment of a cartridge according to the invention as referred to in Example 2;

FIG. 7 shows a perspective view of an embodiment of a reader according to the invention as referred to in Example 2;

FIG. 8 shows a front view of the cartridge inserted in to the reader as referred to in Example 2;

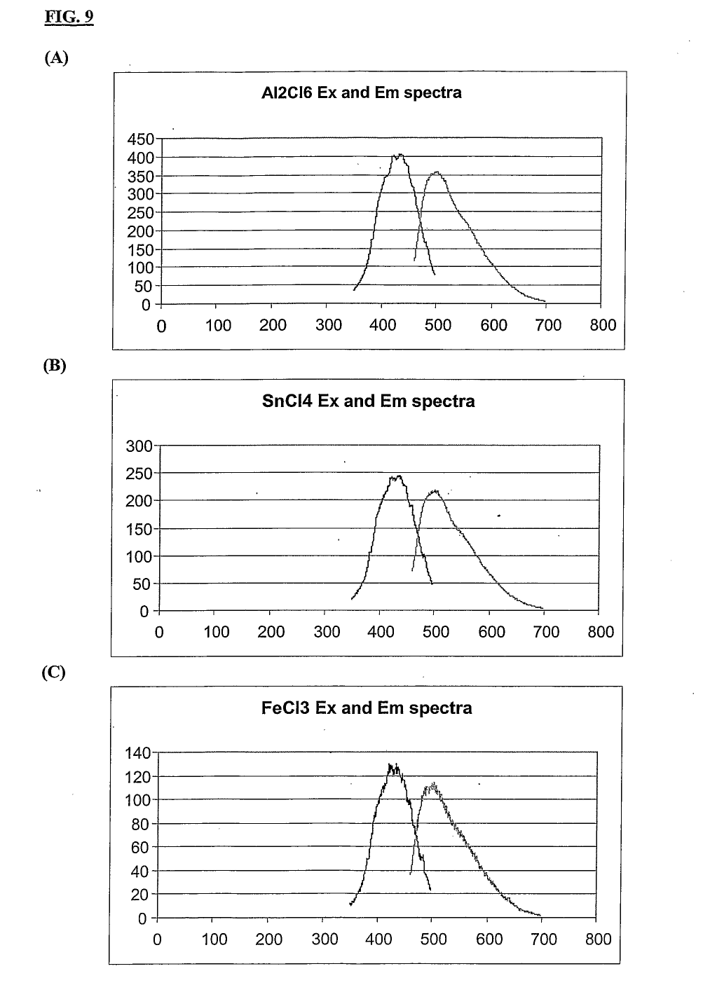

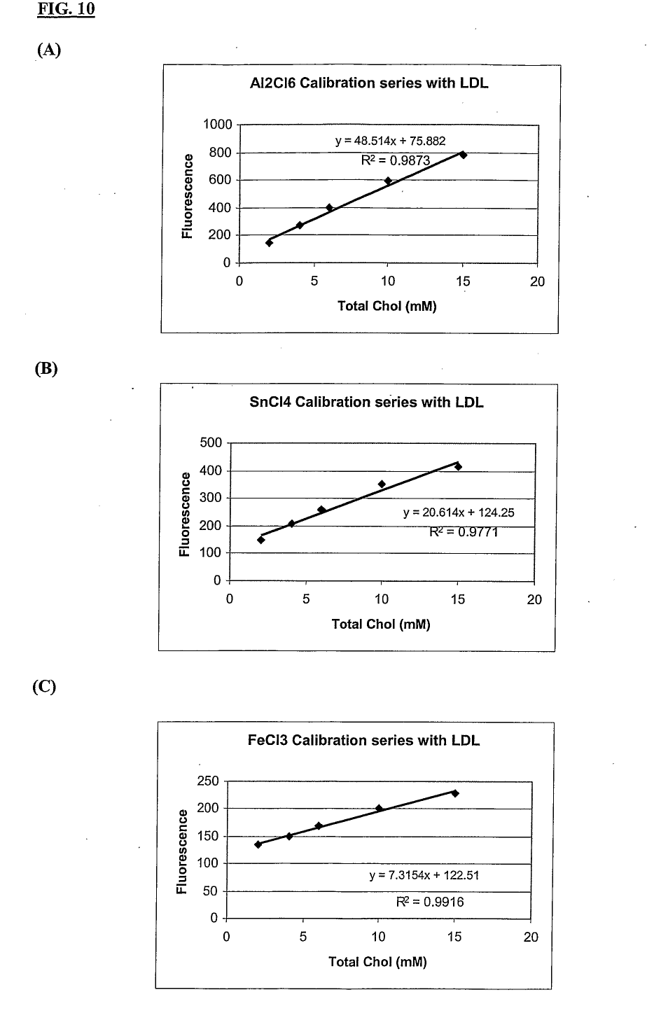

FIG. 9 illustrates the excitation spectra (Ex—dark trace) for the light source and emission spectra (Em—lighter trace) for products of the L-B Assay when the Lewis acids Al₂Cl₆ (A); SnCl₄ (B) and FeCl₃ (C) were utilised in the method of the invention as referred to in Example 3; and

FIG. 10 represents calibration graphs showing fluorescence intensity against the concentration standard cholesterol (in the form of LDL) when utilising Lewis acids, Al₂Cl₆ (A); SnCl₄ (B) and FeCl₃ (C) instead of sulphuric acid in the L-B reaction respectively as referred to in Example 3.

The inventors carried out a series of experiments in order to investigate the use of fluorescence analysis to determine the concentration of cholesterol. Knowledge of the levels of cholesterol in the sample, would be advantageous in helping a clinician decide upon a particular course of treatment. The results of these experiments, which are described in the following examples, were then used to develop the method and apparatus according to the invention.

### Example 1

### Measurement of Total Cholesterol

The inventors investigated whether it would be possible to use fluorescence measurements to determine the concentration of total cholesterol in a sample. This would be in contrast to measuring absorbance as in the conventional assay for cholesterol.

### Method

The method according to the invention was similar to the conventional Liebermann-Burchard reaction assay (L-B), in that the same reagents are used. FIG. 1 illustrates the Liebermann-Burchard reaction. Referring to FIG. 2, there is shown the absorbance spectrum of the L-B product. It can be seen that the absorbance spectrum shows a broad absorbance range, between 400 and 700 nm, which is why absorbance measurements are made with conventional assays to measure cholesterol concentrations in a sample.

However, instead of measuring the absorbance of the coloured product at 550 nm or 600 nm as in the conventional L-B assay and its variations, in the present invention, fluorescence is measured. Referring to FIG. 3, there is shown the fluorescence emission spectrum of the L-B reaction product. Excitation at wavelengths that are responsible for the colour (i.e. 550 nm to 700 nm) do not contribute to this fluorescence. However, when an excitation wavelength of 450 nm was selected, the fluorescence surprisingly extends over the range 470-600 nm.

The advantages of measuring fluorescence instead of measuring absorbance are increased sensitivity and decreased volume requirement. The modified procedure presently uses 50 microlitres of plasma and 1 ml of reagent. This is because a conventional 1 cm pathlength cuvette can be used for the fluorescence measurement. However, reagent volumes in the region of ten microlitres would easily be possible. This could not be achieved using absorbance, as the cell pathlength would be too short for accurate measurements (an absorbance of at least 0.01 Au would be required for accuracy). Unless otherwise indicated, fluorescence measurements were carried out in a Perkin-Elmer LS-50 luminescence spectrometer.

### Measurement of Total Cholesterol

Calibration standards with total cholesterol ranging between 0 and 20 mM were made up from standard, characterised LDL samples. 50 microlitres of a sample were added to 1 ml of L-B reaction reagent, and incubated for 5 minutes at room temperature (although a shorter or longer incubation time may be sufficient for a successful measurement). Fluorescence of each sample was measured using an excitation wavelength of 450 nm, and an emission wavelength of 540 mm. Fluorescence was plotted against total cholesterol concentration as illustrated in FIG. 4. The closeness of the correlation coefficient R² to 1 shows the high degree of linearity of the measurement.

The gradient of the fitted line was used to calculate total cholesterol from the fluorescence of each test sample. Percentage errors between actual and measured total cholesterol are shown in FIG. 5. The results show that the fluorescence L-B reaction assay can be used for measurement of serum cholesterol with very high accuracy in the range from zero to 20 mM, which covers the range that would be expected from clinical samples.

Therefore, the inventors realised that it would be possible to excite a blood sample at 450 nm, and measure the emission at 540 nm, in order to determine the concentration of cholesterol.

### Example 2

### Assay to Determine Concentration of Cholesterol

Example 1 above describes how fluorescent measurements (i.e. not absorbance) of the product of the L-B reaction assay may be used to determine the concentration of total cholesterol in the sample. The inventors observed that the product of the cholesterol assay using the reagents normally used in the L-B assay may be excited at a wavelength of 450 nm and its emission may also be measured at 540 nm. Therefore, the inventors realised that it is possible to devise a method for analysing the concentration of cholesterol of a patient's blood sample using fluorescence analysis.

### Method

A blood sample is initially taken from a patient, and then centrifuged using well-established conventional techniques, in order to separate the serum. Alternatively a blood sample may be filtered to separate cells from serum. The serum is then separated in to a 1 ml aliquot, which is then subjected to biochemical analysis to determine the concentration of cholesterol in the sample, as described below.

25 microlitre of blood serum was added to 2 ml of L-B reaction reagents (60% acetic anhydride, 30% acetic acid and 10% sulphuric acid). The sample was then excited at 450 nm in order to cause the product of the L-B reaction to fluoresce. The fluorescence was measured at an emission wavelength of 540 nm, and from this value it was then possible to determine the concentration of cholesterol in the sample as described in Example 1 above.

### Example 3

### A Device for Calculating the Concentration of Cholesterol

The inventors realised that the method according to the invention, add in particular the small volumes of reagents required for accurate analysis, makes it possible to design a portable apparatus according to the second aspect of the invention comprising a reader and cartridge.

Referring to FIGS. 6-8, there is shown a portable device developed by the inventors, which can be used to calculate the concentration of total cholesterol in a patient's blood sample. The device consists of a cartridge 1, which is shown in detail in FIG. 6, and a reader 50, which is shown in detail in FIG. 7.

The cartridge 1 has a series of interconnected reservoirs, along which fluids may flow in order to carry out the assay according to the invention. The cartridge 1 plugs into the reader 50 via slot 52 for detecting and measuring fluorescence intensity for the assay carried out in the cartridge 1.

Referring to FIG. 6, the cartridge 1 has a sample reservoir 2 in which a biological fluid taken from a patient, such as blood, is contained. A filter 18 may be provided for removing blood cells from the blood, leaving plasma or serum or other body fluid, with which the assay is carried out. The fluid is urged along channels in to reaction reservoir 6, in which the assay is carried out.

Reagent reservoir 14 contains the reagents for the L-B reaction assay, and so when these are urged in to reaction reservoir 6, they are mixed with the biological fluid, and the cholesterol assay (the L-B reaction) is initiated. The cartridge may also include a fluorescence standard 22 for calibrating the reader 50.

In one embodiment of the apparatus (i.e. the cartridge 1 and the reader 50), the assay is carried out in reaction reservoir 6. The cartridge 1 plugs into the slot 52 in the front of the reader 50, as shown in FIG. 8. Slotting the cartridge 1 in to the reader 50 causes the reaction reservoir 6 to align with a corresponding light source 32, and a corresponding detection photodiode 38, which are present in the reader 50.

The light source 32 may take the form of LED (or guide from the LED) and provides the L-B reaction assay with the required fluorescence excitation illumination for the assay products to fluoresce. The wavelength of the light from the LED 32 is at about 450 nm. It may pass through a 450 nm interference filter (not shown) before it is directed to the reaction reservoir 6. The reader 50 has an excitation correction system 46. Hence, fluorescence of the assay is collected with lenses or similar collection optics, and may pass through a polariser at a wavelength of 540 nm. For measurement of the fluorescence intensity, the current output of the photodiode 38 is amplified and read as a current or a voltage.

Accordingly, the reader 50 is configured to hold the cartridge 1 to detect and measure the fluorescence intensities of the assay, to thereby determine the total cholesterol concentration. In one embodiment, the apparatus has an LCD readout display 42 on which the concentration of cholesterol is shown. In another embodiment the reader 50 may be powered by, and have its readout fed through, a USB port of a PC, laptop, or PDA 26 enabling the clinician to readout information on the concentration of cholesterol. Alternatively, the apparatus may embody both aspects of the cartridge 1 and reader 50 and comprise a microprocessor 44 which can calculate the concentrations of cholesterol itself automatically.

Advantages of the cartridge 1 and reader device 50 reside in the quick and easy assay system using the L-B reaction reagents, which can be carried out to determine the concentration of cholesterol. The cartridge 1 is disposable and may be cheaply made being prepared with the L-B reagents for the assay.

### Example 4

### Assays Conducted with Different Strong Acids

The inventors repeated the experiments described in Examples 1 and 2 to illustrate that strong acids, other than sulphuric acid, may be used to measure cholesterol concentrations in a sample according to the invention. The inventors therefore conducted experiments utilising Lewis acids.

The inventors first studied the spectra produced from products of the L-B assay in which the Lewis acids, Al₂Cl₆, SnCl₄ and FeCl₃ were used in the reaction instead of sulphuric acid. The final concentration of each acid was 1.8M (as for the sulphuric acid in Examples 1 and 2). FIG. 9 illustrates the excitation spectra (Ex—dark trace) for the light source and emission spectra (Em—lighter trace) for products of the L-B Assay when the Lewis acids Al₂Cl₆ (A); SnCl₄ (B) and FeCl₃ (C) were used. The emission spectra were equivalent to that obtained when utilising sulphuric acid. This illustrates that other strong acids, in this case Lewis acids, may be utilised in the L-B assay when conducting assays according to the invention.

FIG. 10 represents calibration graphs showing fluorescence intensity against standard cholesterol concentrations (in the form of LDL) when utilising Lewis acids, Al₂Cl₆ (A); SnCl₄ (B) and FeCl₃ (C) instead of sulphuric acid in the L-B reaction. The linearity of these graphs illustrate that reliable measurements of cholesterol concentration can be obtained when a Lewis acid is use in the L-B reaction according to the method of the invention.

1. A method of determining the concentration of total cholesterol in a sample, the method comprising the steps of:
(i) adding to the sample reagents which cause the Liebermann-Burchard (L-B) reaction; and
(ii) determining the total cholesterol concentration in the sample using fluorescence analysis.

2. A method according to claim 1, wherein the sample comprises a biological fluid.

3. A method according to either claim 1 or claim 2, wherein the sample comprises blood serum or plasma, or lymph.

4. A method according to any preceding claim, wherein the reagents added to the sample result in the hydrolysis of all of the total cholesterol present in the sample, i.e. the cholesterol and esters thereof, which may be associated with lipoproteins (e.g. LDL or HDL), which are present in the sample.

5. A method according to any preceding claim, wherein the reagents reduce cholesterol in the sample by adding double bonds thereto.

6. A method according to any preceding claim, wherein the reagents comprise a cholesterol solvent, acetic anhydride, and sulphuric acid.

7. A method according to any preceding claim, wherein the reagents comprise additives, which are used to stabilise impurities in the reagents.

8. A method according to claim 7, wherein the additives comprise anhydrous sodium sulphate, or sodium salicylate.

9. A method according to any preceding claim, wherein the fluorescence is induced by exciting the sample (i.e. the product of the L-B reaction) at an excitation wavelength below about 500 nm, and the resultant fluorescence is measured at an emission wavelength of between 500-650 mm.

10. A method according to claim 9, wherein the fluorescence is induced by exciting the sample at an excitation wavelength of about 450 nm, and the resultant fluorescence is measured at an emission wavelength of about 540 nm.

11. An apparatus for determining the concentration of total cholesterol in a sample, the apparatus comprising: a reaction reservoir for conducting an L-B assay; containment means adapted to contain reagents required for conducting an L-B assay; means for combining the sample and reagents in the reservoir; excitation means operable to excite the sample so that it fluoresces, and detection means operable to detect the fluorescence emitted by the sample.

12. The apparatus according to claim 11 wherein the apparatus comprises a reader and a cartridge and wherein the cartridge comprises the reaction reservoir and the containment means.

13. The apparatus according to claim 12 wherein the reader comprises the excitation means and detection means; and wherein the cartridge comprises a sample reservoir, the containment means in the form of a reagent reservoir, and channels connecting the sample reservoir and reagent reservoir to the reaction reservoir.

14. The apparatus according to any one of claims 11 to 13, wherein the apparatus comprises processing means adapted to determine the concentration of total cholesterol in the sample based on the fluorescence detected.

15. The apparatus according to any one of claims 11 to 14, wherein the apparatus comprises display means for displaying the concentration of total cholesterol in the sample.

16. The apparatus according to any one of claims 11 to 15, wherein the excitation means comprises an illumination source operable to illuminate the sample at about 400 nm-500 nm.

17. The apparatus according to any one of claims 11 to 16, wherein the detection means detects fluorescence emitted by the sample at between 500 nm-650 nm.

18. A cartridge as defined by claim 12 or 13.
